(19) United States
(12) Patent Application Publication
(10) Pub. No.: US 2025/0271405 A1
(43) Pub. Date: Aug. 28, 2025

(54) METHOD FOR ASSISTING WITH NON-TARGETED SCREENING USING DIAGNOSITC FRAGMENTS AND DIAGNOSTIC FRAGMENT GROUPS

(71) Applicant: Peking University, Beijing (CN)

(72) Inventors: Weiling SUN, Beijing (CN); Yi LIU, Beijing (CN); Wen LIU, Beijing (CN); Ting WANG, Beijing (CN); Huazhang ZHAO, Beijing (CN)

(21) Appl. No.: 18/821,178

(22) Filed: Aug. 30, 2024

(30) Foreign Application Priority Data
Feb. 27, 2024 (CN) 202410210612.7

(51) Int. Cl. G01N 30/86; G01N 30/02; G01N 30/72

(52) U.S. Cl. CPC G01N 30/8631; G01N 30/7206; G01N 2030/025

(57) ABSTRACT

Provided is a method for assisting with non-targeted screening using diagnostic fragments and diagnostic fragment groups. The method includes: acquiring a preliminary identification result of compounds contained in a sample, where the preliminary identification result is obtained by analyzing the sample using gas chromatography-high-resolution mass spectrometry and matching peaks after deconvolution with National Institute of Standards and Technology (NIST) library; selecting, from the preliminary identification result, compounds meeting a preset matching condition and containing diagnostic fragments or diagnostic fragment groups to obtain a plurality of initial compounds, where the diagnostic fragments and the diagnostic fragment groups are extracted from a target category of compounds by joint use of a chemical information database and the NIST library; and selecting, from the plurality of initial compounds, an initial compound having the highest comprehensive score as a screening result of the sample.

### CROSS REFERENCE TO RELATED APPLICATION

This patent application claims the benefit and priority of Chinese Patent Application No. 202410210612.7, filed with the China National Intellectual Property Administration on Feb. 27, 2024, the disclosure of which is incorporated by reference herein in its entirety as part of the present application.

### TECHNICAL FIELD

The present disclosure relates to the technical field of organic pollutant screening, and in particular, to a method for assisting with non-targeted screening using diagnostic fragments and diagnostic fragment groups.

### BACKGROUND

Types of organic pollutants are complicated. Traditional targeted analysis focuses on a narrow set of substances, making it challenging to detect new pollutants not previously studied. This approach may overlook major pollutants in complex mediums due to its limited scope. There is a need for non-targeted comprehensive screening methods. Therefore, new pollutant screening based on high-resolution mass spectrometry is a hotspot and leading edge of research. Gas chromatography-high-resolution mass spectrometry has been widely applied to high-throughput screening of volatile and semi-volatile organic compounds. However, for a category of organic pollutants or organic pollutants having a specific structure, how to carry out non-targeted screening is still a huge challenge. At present, the primary method for identifying the structure of non-targeted substances in non-target screening using gas chromatography-high-resolution mass spectrometry is to compare results with the National Institute of Standards and Technology (NIST) library. However, this method has certain limitations. When comparing a mass spectrum of a substance with the NIST library, multiple possible substances are often identified, and the substance with the highest comprehensive score may not necessarily be the correct identification. Therefore, in addition to comparison with the NIST library, there is an urgent need to develop additional methods to assist with structural identification of non-targeted substances, thereby improving the accuracy of non-targeted screening results.

Diagnostic fragment analysis is a common method used in non-targeted screening processes. However, existing methods disclosed in patents such as CN116718715A and CN116297923A still have limitations in their diagnostic fragment extraction processes. These patents disclose extracting diagnostic fragments based on specific fragmentation patterns and subsequently screening for a particular class of substances. Although these methods demonstrate screening effectiveness for specific class of substances, they require a well understanding of the fragment patterns. Moreover, their practical utility is often constrained by reliance on personal experience or specialized knowledge, making them less convenient to promote and use widely. A universal diagnostic fragment method is still to identify fragments that consistently appear with high abundance and frequency in the spectra of a specific category of target pollutants. Therefore, when screening diagnostic fragments, it is vital to use as many spectra as possible as data sources.

At present, most diagnostic fragment screening methods are based on actually measured spectra (CN115753953A), and have the disadvantages of limited standard samples, high costs, a limited number of spectra acquired, and time-consuming and labor-consuming test sample extraction processes. Existing parent (CN113009054B) proposes collecting data from the NIST library and extracting diagnostic fragments. Compared with methods solely based on actually measured spectra, this method expands the range of substances during diagnostic fragment screening to a certain extent. However, when this method is used in determining vanillin type flavor and fragrance substances, 27 compounds structurally similar to vanillin are found in a list of flavors and fragrances allowed to be added in GB 2760-2014, and fragment ions and abundances of these compounds are obtained from NIST library and standard substance detection. Relying solely on existing data to determine a comprehensive list of compounds within a specific class of flavors and fragrances has inherent limitations due to current knowledge gaps and study constraints. This approach can easily overlook certain compounds that may not have been thoroughly researched or cataloged in existing databases. Moreover, the current screening method for diagnostic fragments relies solely on a single fragment, which may lead to false positives.

### SUMMARY

An objective of the present disclosure is to provide a method for assisting with non-targeted screening using diagnostic fragments and diagnostic fragment groups that can improve the accuracy of a non-targeted screening result.

To achieve the above objective, the present disclosure provides the following solutions: a method for assisting with non-targeted screening using diagnostic fragments and diagnostic fragment groups includes: acquiring a preliminary identification result of compounds contained in a sample, where the preliminary identification result is obtained by analyzing the sample using gas chromatography-high-resolution mass spectrometry and matching all peaks after deconvolution with National Institute of Standards and Technology (NIST) library;
selecting, from the preliminary identification result, compounds meeting a preset matching condition and containing diagnostic fragments or diagnostic fragment groups to obtain a plurality of initial compounds, where the diagnostic fragments and the diagnostic fragment groups are extracted from a target category of compounds by joint use of a chemical information database and the NIST library; and
selecting, from the plurality of initial compounds, an initial compound having the highest comprehensive score as a screening result of the sample.

Optionally, the diagnostic fragments and the diagnostic fragment groups may be extracted by: retrieving a Chemical Abstracts Service (CAS) number of a target compound molecule in the chemical information database, where a common structure of a target category of chemicals is used as a substructure of the target compound molecule;
performing batch searching in a NIST mass spectral database according to the CAS number to obtain mass spectral data of a plurality of compounds;
extracting fragment ions from the mass spectral data of the compounds to obtain a plurality of fragments, where the fragment ions are fragments meeting a preset requirement in the mass spectra of the compounds;
selecting diagnostic fragments from the fragment ions according to an occurrence frequency of each fragment ions, where the occurrence frequency of the diagnostic fragment is higher than that of other fragment ions excluding the diagnostic fragments; and
selecting diagnostic fragment groups from the fragment ions according to an occurrence law of each of the fragment ions, where the diagnostic fragment group includes at least two fragment ions and each fragment ion in the diagnostic fragment group must occur in the plurality of compounds.

Optionally, the selecting the diagnostic fragment from fragment ions may specifically include: selecting, from the fragment ions arranged in order of occurrence frequency from high to low, a first preset number of fragment ions to obtain a plurality of diagnostic fragments.

Optionally, the selecting a diagnostic fragment group from the fragment ions according to an occurrence law of each of the fragment ions may specifically include: counting the fragment ions occurring in the compounds to obtain a plurality of initial diagnostic fragment groups; and
selecting, from the initial diagnostic fragment groups arranged in order of occurrence frequency from high to low, a second preset number of initial diagnostic fragment groups to obtain a plurality of diagnostic fragment groups.

Optionally, the retrieving a CAS number of a target compound molecule in the chemical information database may specifically include: retrieving the target compound molecule in the chemical information database; and
extracting the CAS number of the target compound molecule according to compound molecule information of the target compound molecule.

Optionally, the retrieving the target compound molecule in the chemical information database may specifically include: retrieving a common structure of a target category of chemicals in the chemical information database; and
collecting the target compound molecule having the common structure as a substructure.

Optionally, a format of the mass spectral data of each of the compounds is a Mass Spectral Peaks (MSP) format.

According to the specific embodiments provided in the present disclosure, the present disclosure has the following technical effects: the method of the present disclosure includes: acquiring a preliminary identification result of compounds contained in a sample, and selecting compounds meeting a preset matching condition and containing diagnostic fragments or diagnostic fragment groups to obtain a plurality of initial compounds, where the diagnostic fragment sand the diagnostic fragment groups are extracted from a target category of compounds by joint use of a chemical information database and the NIST library; and finally, selecting, from the initial compounds, an initial compound having the highest comprehensive score as a screening result of the sample. By extracting the diagnostic fragments from the target category of compounds using the chemical information database and the NIST library in combination, the present disclosure can overcome the defect that a substance having the highest comprehensive score among a plurality of possible substances obtained by the method of comparison with the NIST library is not really the true result. Moreover, in addition to comparison with the NIST library, the concepts of the diagnostic fragment and the diagnostic fragment group are further proposed. Further, the chemical information database and the NIST library are used in combination to extract the diagnostic fragment and the diagnostic fragment group from the target category of compounds. The diagnostic fragment and the diagnostic fragment group are used to assist with structural identification of a non-targeted substance, thereby improving the accuracy of a non-targeted screening result.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a flowchart of a method for assisting with non-targeted screening using diagnostic fragments and diagnostic fragment groups provided by Example 1 of the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments derived from the embodiments in the present disclosure by a person of ordinary skill in the art without creative efforts shall fall within the protection scope of the present disclosure.

An objective of the present disclosure is to provide a method for assisting with non-targeted screening using diagnostic fragments and diagnostic fragment groups that can improve the accuracy of a non-targeted screening result.

To make the above objectives, features, and advantages of the present disclosure clearer and more comprehensible, the present disclosure will be further described in detail below with reference to the accompanying drawings and the specific examples.

Example 1: a method for assisting with non-targeted screening using diagnostic fragments and diagnostic fragment groups in this Example includes step 1 to step 3.

In step 1, a preliminary identification result of compounds contained in a sample is acquired, where the preliminary identification result is obtained by scanning the sample using gas chromatography-high-resolution mass spectrometry and matching all peaks after deconvolution with National Institute of Standards and Technology (NIST) library.

In step 2, compounds meeting a preset matching condition and containing diagnostic fragments or diagnostic fragment groups are selected from the preliminary identification result to obtain a plurality of initial compounds, where the diagnostic fragments and the diagnostic fragment groups are extracted from a target category of compounds by joint use of a chemical information database and the NIST library.

In step 3: an initial compound having a highest comprehensive score is selected from the initial compounds as a screening result of the sample.

A process of extracting the diagnostic fragments and the diagnostic fragment groups specifically includes step 2.1 to step 2.5.

In step 2.1, a Chemical Abstracts Service (CAS) number of a target compound molecule is retrieved in the chemical information database, where a common structure of a target category of chemicals is used as a substructure of the target compound molecule.

Step 2.1 specifically includes step 2.1.1 to step 2.1.2.

In step 2.1.1, the target compound molecule is retrieved in the chemical information database.

Step 2.1.1 specifically includes step 2.1.1.1 to step 2.2.1.2.

In step 2.1.1.1, a common structure of a target category of chemicals is retrieved in the chemical information database.

In step 2.1.1.2, the target compound molecule having the common structure as a substructure is collected.

In step 2.1.2, the CAS number of the target compound molecule is extracted according to compound molecule information of the target compound molecule.

In step 2.2, batch searching is performed in a NIST mass spectral database according to the CAS number to obtain mass spectral data of a plurality of compounds.

Specifically, a format of the mass spectral data of each of the compounds is a Mass Spectral Peaks (MSP) format.

In step 2.3, fragment ions are extracted from the mass spectral data of the compounds to obtain a plurality of fragments, where the fragment ions are fragments meeting a preset requirement in the mass spectral data of the compounds.

In step 2.4, a diagnostic fragment is selected from a plurality of fragment ions according to an occurrence frequency of each of the fragment ions, where the occurrence frequency of the diagnostic fragment is greater than occurrence frequencies of other fragment ions than the diagnostic fragment.

Specifically, a first preset number of fragment ions are selected from the fragment ions arranged in order of occurrence frequency from high to low to obtain a plurality of diagnostic fragments.

In practical use, a specific value of the first preset number may be selected according to an actual situation.

In step 2.5, a diagnostic fragment group is selected from the fragment ions according to an occurrence law of each of the fragment ions, where the diagnostic fragment group includes at least two fragment ions and each fragment ion in the diagnostic fragment group must occur in at least one compound.

Specifically, the selecting diagnostic fragment groups from the fragment ions includes: count the fragment ions occurring in the compounds to obtain a plurality of initial diagnostic fragment groups; and select, from the initial diagnostic fragment groups arranged in order of occurrence frequency from high to low, a second preset number of initial diagnostic fragment groups to obtain a plurality of diagnostic fragment groups. In practical use, a specific value of the second preset number may be selected according to an actual situation.

In practical use, individual fragments are screened, equivalently by an occurrence frequency. For the screening of a diagnostic fragment group, in consideration of two (and more) fragments occurring in one compound, the fragments are selected as a diagnostic fragment group when the occurrence frequency thereof is higher than a certain frequency.

The diagnostic fragment group is not obtained by combining screened individual diagnostic fragments. Instead, the diagnostic fragment group is re-extracted. It is required that two (or more than two) fragments must occur in the spectrum of a single compound. For example, the diagnostic fragment group composed of C5H3N and C3HN occurs in the spectra of 46 compounds of the target category. Therefore, the diagnostic fragment group includes not only an occurrence frequency but also an occurrence law (some fragments always co-occur). The purpose of extracting the diagnostic fragment group is as follows: since a single diagnostic fragment may cause a false positive of screening (it is not the target compound studied in the present disclosure, but still contains a certain diagnostic fragment), the diagnostic fragment group can reduce false positives.

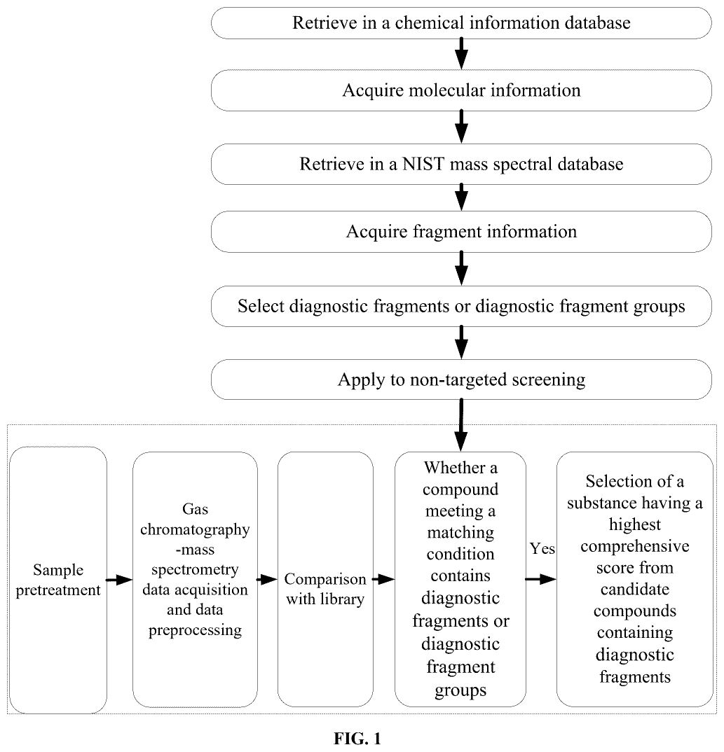

In one specific implementation, as shown in FIG. 1, a specific application process of the method for assisting with non-targeted screening using diagnostic fragments and diagnostic fragment groups includes step S1 to step S6.

In S1, retrieving is performed in the chemical information database. A common parent structure of a category of chemicals is retrieved in the public chemical information database to collect a molecule containing this substructure. Preferably, retrieving is performed in Pubchem chemical information database.

Specifically, the research object of this Example is an aromatic nitrogen heterocycle compound. The aromatic nitrogen heterocycle structure is retrieved in the Pubchem database. Chemical molecules including pyrrole, pyridine, pyrazine, pyridazine, pyrimidine, indole, quinoline, and isoquinoline as common structures are retrieved, and molecules containing the substructures are collected.

In S2, molecular information is acquired. The molecular information of the compounds collected from the chemical information database is downloaded, and CAS numbers thereof are extracted by python programming.

Specifically, the molecular information of the compounds collected from the chemical information database is downloaded, and the CAS numbers thereof are extracted by programming using python. The processed data is as follows: 33 pyrrole compounds, 576 pyridine compounds, 16 pyrazine compounds, 17 pyridazine compounds, 175 pyrimidine compounds, 2283 indole compounds, 1285 quinoline compounds, and 502 isoquinoline compounds, and a total of 4887 aromatic nitrogen heterocycle compounds are retrieved.

In S3, retrieving is performed in a NIST mass spectral database. Batch searching is performed in the NIST mass spectral database using the CAS numbers. Fragment mass and abundance information included in the column Num Peaks in the mass spectral data of the MSP format is downloaded for subsequent statistics.

Specifically, batch searching is performed in the NIST mass spectral database using the CAS numbers. The mass spectral data of the MSP format is downloaded for subsequent statistics of fragment information. The retrieved records having matched NIST mass spectral data are as follows: 2 pyrrole compounds, 46 pyridine compounds, 2 pyrazine compounds, 2 pyridazine compounds, 23 pyrimidine compounds, 40 indole compounds, 116 quinoline compounds, and 31 isoquinoline compounds, and the NIST mass spectral data of 262 aromatic nitrogen heterocycle compounds is collected in total.

In S4, the fragment information is acquired. Programming is performed using python. A mass-to-charge ratio (m/z) of a fragment having a high abundance contained in each substance in MSP is extracted. For the requirement on the abundance, different thresholds may be set according to practical application situations and needs. Preferably, fragment information having first 10 abundances in the mass spectral data of each compound is selected.

Abundance is information included in the column Num Peaks, and a format thereof is as follows: mass number abundance; mass number abundance; mass number abundance . . .

Specifically, programming is performed using python. Fragment mass information having first 10 abundances in the mass spectral data corresponding to 262 compounds in MSP is extracted.

In S5, diagnostic fragments and diagnostic fragment groups are selected.

The occurrence frequencies of the fragments are counted. A fragment having a high occurrence frequency is selected as the diagnostic fragment, and a molecular formula is inferred according to a m/z thereof. Preferably, the fragments having first 10 total occurrence frequencies are used as the diagnostic fragments.

Further, inferring the molecular formula according to the m/z is specifically as follow: the elementary composition of the diagnostic fragment may be inferred in combination with the elementary composition of the target organic matter. For example, in an embodiment, elements included and exact masses thereof are as follows: C=12.0000, H=1.0078, and N=14.0064. For example, the selected fragment having the highest occurrence frequency is as follows: 51=C(12)×3+N (14)×1+H (1)×1. When C3NH is determined, its exact mass is equal to 12.0000×3+14.0064+1.007. If a diagnostic fragment is composed of a plurality of elements, the molecular composition of the diagnostic fragment may be determined in combination with methods such as previous research experience, inferring according to the fragmentation law, checking in standard samples, and verifying in an actual mass spectrum.

Particular fragment combinations (2 and more) having high occurrence frequencies are selected as diagnostic fragment groups, and the exact mass is calculated according to the m/z of each fragment in the diagnostic fragment group. Preferably, the diagnostic fragment combinations occurring at least in the mass spectral data of 20 compounds are used as the diagnostic fragment groups.

Specifically, the fragments having first 10 total occurrence frequencies are used as the diagnostic fragments. The diagnostic fragment groups occurring at least in the mass spectral data of 20 compounds are counted. The molecular formula of the diagnostic fragment is inferred according to the m/z thereof. The diagnostic fragments of nitrogen heterocycle compounds extracted from the NIST spectra of 262 aromatic nitrogen heterocycle compounds are as shown in Table 1, and the diagnostic fragment groups are as shown in Table 2.

TABLE 1
Record Table of Diagnostic fragments of
Aromatic Nitrogen Heterocycle Compounds

| | | Molecular | |
|---|---|---|---|
| Mass-to-charge | Count of | Composition | Extract |
| ratio, m/z | Occurrences | of Fragment | m/z |
| 51 | 112 | C3HN | 51.01035 |
| 77 | 64 | C5H3N | 77.026 |
| 50 | 64 | C3N | 50.00253 |
| 63 | 60 | C4HN | 63.01035 |
| 115 | 57 | C8H5N | 115.04165 |
| 142 | 56 | C10H8N | 142.06513 |
| 39 | 53 | C2HN | 39.01035 |
| 143 | 52 | C10H9N | 143.07295 |
| 128 | 51 | C9H6N | 128.04948 |
| 89 | 48 | C6H3N | 89.026 |

TABLE 2
Record Table of Diagnostic fragment groups
of Aromatic Nitrogen Heterocycle Compounds

| Diagnostic | Count of |
|---|---|
| Fragment Groups | Occurrences |
| C5H3N , C3HN | 46 |
| C10H9N, C2HN | 31 |
| C10H8N, C10H9N | 27 |
| C3HN, C3N | 22 |

In S6, the diagnostic fragments and the diagnostic fragment groups are applied to non-targeted screening.

Steps of applying the diagnostic fragments and the diagnostic fragment groups to non-targeted screening include: sample pretreatment, gas chromatography-mass spectrometry data acquisition and data preprocessing, comparison with library, determination whether there are candidate compounds meeting a matching condition and containing the diagnostic fragment groups or the diagnostic fragments, and selection of a substance having a highest comprehensive score from the candidate compounds containing the diagnostic fragment groups or the diagnostic fragments as a final result. One or more fragments having a particular exact mass may be directly searched in spectrum processing software to select substances containing only the diagnostic fragments. The spectral result of each substance may also be checked directly. Specifically, the comprehensive score is acquired by spectrum processing software, such as GC Deconvolution, TraceFinder, and Compound Discoverer.

In practical use, suitable pretreatment manners are selected for different sample properties and experimental requirements, including, but not limited to, liquid-liquid extraction, solid-phase extraction, Soxhlet extraction, ultrasonic extraction, and the like. The sample type in this Example is industrial wastewater. The sample pretreatment is performed using the liquid-liquid extraction method. Data is acquired from the data using gas chromatography-high-resolution mass spectrometry. Instrument parameters are set. After data acquisition, deconvolution processing and matching of candidate compounds are performed using software. In this Example, data is acquired using GC-Orbitrap. A total of 946 peaks are obtained after deconvolution.

After matching with the NIST library and GC-Orbitrap self-built pollutant library provided by a provider, in a candidate list matched with each peak, matching results that candidate aromatic nitrogen heterocycle compounds meet the matching conditions of RSI>600, RHRMF>75%, and ΔRI<50, and contain at least one diagnostic fragment in Table 1 are retained. A total of 10 peaks meeting the conditions are obtained.

For the resulting target peaks and candidate matching results, the candidate compound of the target category having the highest comprehensive score is selected as the matching result. In this Example, the information of nitrogen heterocycle substances contained in an industrial waste water sample and comprehensive score results thereof are as shown in Table 3.

TABLE 3
Statistical Table of Information of Nitrogen Heterocycle Substances in
Industrial Waste Water Sample and Comprehensive Score Results Thereof

| | Retention | Structural | | Comprehensive |
|---|---|---|---|---|
| Substance Name | Time | Formula | CAS | Score |
| Pyridine,4-methyl- | 3.724 | C6H7N | 108-89-4 | 97.5 |
| Pyrazine,ethyl- | 5.666 | C6H8N2 | 13925-00-3 | 96.7 |
| Pyridine,2,5-dimethyl- | 6.154 | C7H9N | 589-93-5 | 94.5 |
| 2-Pyridineethanol | 8.684 | C7H9NO | 103-74-2 | 93.3 |
| Benzothiazole | 14.089 | C7H5NS | 95-16-9 | 96.4 |
| Deferiprone | 14.231 | C7H9NO2 | 30652-11-0 | 94.9 |
| Thieno[2,3-c]pyridine | 14.798 | C7H5NS | 272-12-8 | 97.7 |
| Thieno[2,3-b]pyridine | 14.937 | C7H5NS | 272-23-1 | 96.1 |
| Quinoline,3-methyl- | 17.396 | C10H9N | 612-58-8 | 94.6 |
| 2,4(1H,3H)-Quinolinedione,3- | 22.543 | C16H21NO3 | 69808-30-6 | 94.8 |
| heptyl-3-hydroxy- | | | | |

Compared with the prior art, the present disclosure has the following beneficial effects: 1. The method the present disclosure relates to is widely applicable. When the method of the present disclosure is used, only the common structure of the studied type of chemicals needs to be determined. It is not required to know the fragmentation law of the target category of chemicals, include a list of substances, or detect a standard sample.
2. The present disclosure has the advantages of comprehensive spectrum retrieval and high reliability of the resulting diagnostic fragments. For organic pollutants having a certain common parent structure, the chemical database and the NIST library are used in combination to comprehensively retrieve the structure and spectrum information of a particular category of organic pollutants. On this basis, the resulting diagnostic fragments and diagnostic fragment groups are comprehensive and reliable.
3. The present proposes the concept and the extraction method of the diagnostic fragment group. That is, in addition to extracting a single diagnostic fragment having a high occurrence frequency, extracting the diagnostic fragment group occurring in different compound spectra for a plurality of times is also considered. The diagnostic fragments and the diagnostic fragment groups are used to assist with structural identification of non-targeted substances. False positive of screening that may be possibly caused by a single diagnostic fragment can be overcome. The accuracy and reliability of the screening result are improved.
4. In the diagnostic fragment screening process of the present disclosure, data extraction may be completed automatically using a code rather than manually. Simple use, and time and labor saving are achieved.

The technical features of the foregoing embodiments can be combined arbitrarily. For brevity of description, not all possible combinations of the technical features of the foregoing embodiments are described. However, the combinations of these technical features should be construed as falling within the scope described in this specification as long as there is no contradiction between the combinations.

Specific examples are used herein to explain the principles and implementations of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of this specification shall not be construed as limitations to the present disclosure.

What is claimed is:

1. A method for assisting with non-targeted screening using diagnostic fragments and diagnostic fragment groups, comprising:
acquiring a preliminary identification result of compounds contained in a sample, wherein the preliminary identification result is obtained by analyzing the sample using gas chromatography-high-resolution mass spectrometry and matching all peaks after deconvolution with National Institute of Standards and Technology (NIST) library;
selecting, from the preliminary identification result, compounds meeting a preset matching condition and containing diagnostic fragments or diagnostic fragment groups to obtain a plurality of initial compounds, wherein the diagnostic fragments and the diagnostic fragment groups are extracted from a target category of compounds by joint use of a chemical information database and the NIST library; and
selecting, from the plurality of initial compounds, an initial compound having a highest comprehensive score as a screening result of the sample.

2. The method for assisting with non-targeted screening using diagnostic fragments and diagnostic fragment groups according to claim 1, wherein the diagnostic fragments and the diagnostic fragment groups are extracted by:
retrieving a Chemical Abstracts Service (CAS) number of a target compound molecule in the chemical information database, wherein a common structure of a target category of chemicals is used as a substructure of the target compound molecule;
performing batch searching in a NIST mass spectral database according to the CAS number to obtain mass spectral data of a plurality of compounds;
extracting fragment ions from the mass spectral data of the compounds to obtain a plurality of fragments, wherein the fragment ions are fragments meeting a preset requirement in the mass spectral data of the compounds;
selecting diagnostic fragments from the fragment ions according to an occurrence frequency of each of the fragment ions, wherein the occurrence frequency of the diagnostic fragment is higher than that of other fragment ions excluding the diagnostic fragments; and
selecting diagnostic fragment groups from the fragment ions according to an occurrence law of each of the fragment ions, wherein the diagnostic fragment group comprises at least two fragment ions and each fragment ion in the diagnostic fragment group occurs in the plurality of compounds.

3. The method for assisting with non-targeted screening using diagnostic fragments and diagnostic fragment groups according to claim 2, wherein the selecting the diagnostic fragment from fragment ions specifically comprises:
selecting, from the fragment ions arranged in order of occurrence frequency from high to low, a first preset number of fragment ions to obtain a plurality of diagnostic fragments.

4. The method for assisting with non-targeted screening using diagnostic fragments and diagnostic fragment groups according to claim 2, wherein the selecting diagnostic fragment groups from the fragment ions according to an occurrence law of each of the fragment ions specifically comprises:
counting the fragment ions occurring in the compounds to obtain a plurality of initial diagnostic fragment groups; and
selecting, from the initial diagnostic fragment groups arranged in order of occurrence frequency from high to low, a second preset number of initial diagnostic fragment groups to obtain a plurality of diagnostic fragment groups.

5. The method for assisting with non-targeted screening using diagnostic fragments and diagnostic fragment groups according to claim 2, wherein the retrieving a CAS number of a target compound molecule in the chemical information database specifically comprises:
retrieving the target compound molecule in the chemical information database; and
extracting the CAS number of the target compound molecule according to compound molecule information of the target compound molecule.

6. The method for assisting with non-targeted screening using diagnostic fragments and diagnostic fragment groups according to claim 5, wherein the retrieving the target compound molecule in the chemical information database specifically comprises:
retrieving a common structure of a target category of chemicals in the chemical information database; and
collecting the target compound molecule having the common structure as a substructure.

7. The method for assisting with non-targeted screening using diagnostic fragments and diagnostic fragment groups according to claim 2, wherein a format of the mass spectral data of each of the compounds is a Mass Spectral Peaks (MSP) format.
